(19) United States
(12) Patent Application Publication
(10) Pub. No.: US 2010/0174580 A1
(43) Pub. Date: Jul. 8, 2010

(54) METHOD FOR INTERDEPARTMENTAL COORDINATION OF SOFTWARE-ASSISTED ACTIVITY, IN PARTICULAR IN A HOSPITAL

(76) Inventors: Karlheinz DORN, Kalchreuth (DE); Vladyslav Ukis, Nurnberg (DE)

Correspondence Address: HARNESS, DICKEY & PIERCE, P.L.C., P.O.BOX 8910, RESTON, VA, 20195, US

(21) Appl. No.: 12/641,408

(22) Filed: Dec. 18, 2009

(30) Foreign Application Priority Data
Jan. 7, 2009 (DE) 10 2009 004 004.8

(51) Int. Cl. G06Q 10/00

(52) U.S. Cl. 705/9; 705/7

(57) ABSTRACT

In a hospital, a virtual team can be formed on an interdepartmental basis, with individual roles of treating physicians being defined based on a scheme appropriate to a treatment situation and specific physicians being allocated to the roles such that just-in-time planning of a patient's treatment can be carried out. In at least one embodiment, the sequential execution of tasks appropriate to the individual departments is monitored by a central data processing device.

### PRIORITY STATEMENT

The present application hereby claims priority under 35 U.S.C. §119 on German patent application number DE 10 2009 004 004.8 filed Jan. 7, 2009, the entire contents of which are hereby incorporated herein by reference.

### FIELD

At least one embodiment of the invention generally relates to a method for coordinating software-assisted activity of a plurality of departments in an organization, the method being provided in particular for a hospital.

### BACKGROUND

In hospitals, many patients pass through a plurality of departments. It has meanwhile become common practice in hospitals to perform activities with software support, e.g. the recording of patient images, the management of the patient images, the viewing of the patient images and finally also the generation of medical reports, with each department having its own software. In the past there has been no coordination of the patient's visit to different departments. Rather it has been the case that if it is deemed necessary to have activities performed by another department, one department transfers the patient to precisely that other department. There, the patient is received effectively like a new patient. Each department operates its own time management. This results in the patients having to put up with waiting times in the individual departments.

Particularly in the case of emergency patients who have to be examined in a number of departments, a quick examination is desirable, if not vital for the patients. It would be desirable if a patient's passage through departments of a hospital could be organized according to the just-in-time principle. The fact that the software for each individual department of a hospital is provided separately makes just-in-time planning impossible. Often individual departments of a hospital even use totally different software systems which are incompatible with one another.

### SUMMARY

At least one embodiment of the invention is directed to a method for coordinating software-assisted activity in a plurality of departments of an organization, in particular a hospital, so that better planning of this activity is made possible. In the case of the hospital it is aimed to enable the patient to be allocated to individual departments according to the just-in-time principle, thereby improving the patient's situation.

The method according to at least one embodiment incorporates the provisioning of software and data, whereas the emphasis in the method according to the second aspect is on the use of software.

The method according to at least one embodiment begins with the step of providing a central data processing device as well as departmental data processing devices coupled to same for the purpose of exchanging data, with at least one such departmental data processing device for each department. Thus, a network having one central point of control is built up in the organization. The method continues with the step of receiving at least one input for designating a possible starting situation. The purpose of this is to enable the activity that is to be coordinated to be designated as a whole. In the step of receiving at least one input for specifying a sequence of functions and roles assigned by the individual functions, it is then specified as a logical consequence, which type of activity (functions) is to be performed according to the designation, i.e. in the starting situation. At the same time, by specifying the roles it is defined from which group of people the individual having to perform an individual function can be selected.

In a hospital, the performance of many a function demands a specific qualification. In this case, in particular the professional qualification (cardiologist, oncologist etc.) can be specified as the role. For each function there is now received in addition at least one input for assigning software task flows. At the same time the software for these software tasks is provided. Let it be noted that what is to be understood by “software task” is the provisioning of a specific functionality. The higher-order concept of the “function”, on the other hand, encompasses the abstract specification of which objective is to be reached, or how an objective is to be reached.

By way of the steps a) to d) of the method according to at least one embodiment that has just been explained, the preconditions are first established that enable software-assisted activity to be coordinated. A distinction is now made in at least one embodiment between two cases: First, the preconditions are tailored to a specific starting situation which was not thought of previously. Then, the inputs must naturally have been made when this starting situation occurs as a real starting situation. In the method according to at least one embodiment the invention, the inputs are then made available in any case to the central data processing device, either directly via input means at the central data processing device, or they are input via input means at a departmental data processing device—e.g. a data processing device in an emergency admissions unit of a hospital—and then, owing to the coupling of same to the central data processing device, transferred to the latter. In the present scenario, namely that the received inputs are directly available on the central data processing device at any event, it is then only necessary for a real starting situation, on the preexistence of which the further course of method is based, to be equated to the possible starting situation.

Alternatively, all steps a) to d) of the method according to at least one embodiment can have been performed in preparation for the occurrence of a real starting situation, steps b) to d) in particular being performed more than once for different possible starting situations. Then the results of the inputs should be provided on the central data processing device. On the basis of the results having been provided, the method then proceeds as follows: An input for designating the real starting situation is received, and this can then be assigned to the corresponding possible starting situations.

In both alternatives cited in step e) of at least one embodiment, a real starting situation is then assigned a function flow, and the latter is in turn assigned a software task flow, with software being available for the software tasks.

For each function from the function flow assigned to the now defined real starting situation, the central data processing device is now placed in readiness to receive an identification input for the role assigned to the function on a departmental data processing device, wherein the departmental data processing device in particular can be assigned to this role. The identification input can be e.g. a conventional login password. Finally, such an identification input is received, and tasks associated with the relevant function for which the identification input was made are generated and the task flow executed. Generating a task essentially consists in software being written into a working memory, which is to say being allowed to run. In the present case the tasks are to be generated and the task flow is to be executed using program code stored on memory of the central data processing device. The central data processing device can therefore assume the function of coordination and process a task flow task by task, and in turn process, task flow by task flow, each function in the function flow. In this case the departmental data processing devices play a supporting role, but are dependent on the central data processing device.

In this way it becomes possible to carry out a full planning of how activity in the organization can be completed, in particular how a patient can be treated step by step in different departments in a hospital. There is no longer any necessity for the individual departments to communicate with one another, in other words e.g. where one department makes a decision about to which other department a patient is to be transferred; instead, the coordination of the activity is accomplished centrally according to a predetermined scheme, associated with the possible starting situation, which has then been identified with a real starting situation. Physically transferring the patient between departments also constitutes a possible function, with the result that this is planned centrally and not left to the respective department pairings.

The software on the central data processing device does not have to effect the execution of tasks on its own; rather, the tasks can be generated through interaction between the central data processing device and a departmental data processing device assigned to the respective role, in other words through interaction between executing program code on the central data processing device on the one hand and the departmental data processing device on the other. The tasks are preferably embodied such that they interact with a user located in the region of the departmental data processing device. Thus, they can act on an output device connected to the departmental data processing device. Alternatively or in addition, they can also respond to an input made on an input device connected to the departmental data processing device.

Via the output device the operator can be prompted to perform certain steps sequentially. This can include the inputting of data which is then processed subsequently. The software is divided up into software components (program code packages), wherein by preference those program code packages that are specifically responsible for the communication between the system and the operator are arranged in the region of the departmental data processing device, whereas in particular such recurring operations of the software that are required as part of the activity of different departments are assigned to the central data processing device. For example, the central data processing device can be responsible for the function of visualizing and storing images; it can then fulfill this function in each case by interacting with different departmental data processing devices. Equally, the central data processing device can administer a medical report into which new entries are inserted in each case by the individual departments.

It is possible to perform the method according to at least one embodiment the invention and leave it to the individual departments to decide which individual and which devices will be used in each case when individual functions are fulfilled. In a preferred embodiment variant, however, individuals and/or devices are allocated to the individual roles in advance by the central data processing device, in other words before step f) of the method according to at least one embodiment of the invention. The allocation can include in particular that a time interval for fulfilling the associated function is additionally assigned to the individuals and/or devices, such that a timeline is defined.

To ensure the timeline is adhered to, the central data processing device then notifies the allocated individuals that they have to fulfill the function. This notification can be communicated simultaneously to all allocated individuals so that they know at an early stage when they have to expect the arrival of a particular patient and which functions they will then have to perform. Alternatively or in addition, the issuing of notifications by the departmental data processing devices to actual individuals can be initiated by the central data processing device at times defined by the timeline. This can include e.g. that in a departmental data processing device the users are called upon to log in under a specific role. The treating physicians are then instructed by the central data processing device to adhere to the planned timeline. This enables just-in-time treatment of a patient, which is helpful in particular in the case of emergency patients.

The method according to at least one embodiment for coordinating software-assisted activity of a plurality of hospital departments comprises the steps of:
a) specifying a treatment situation from a plurality of stored possible treatment situations based on an input,
b) allocating a function flow (relevant to the treatment situation) with roles associated with each function,
c) allocating software task flows to each function, and
d) effecting the execution of tasks on data processing devices in hospital departments differing according to role (in particular based on respective inputs, e.g. logins) in accordance with the allocated function flow and with software task flows assigned to the functions of said function flow.

The input in step a) can be, for example, an identification code under which a specific treatment situation is administered in a central data processing device, and the latter can then access a scheme that is stored in relation to the respective treatment situation and under the scheme can then perform the allocating steps, e.g. by retrieving previously stored files. The input in step a) can also be made by selection from a menu in which different treatment situations are represented. The specification of the treatment situation by the associated data processing device is then effected by the click of a mouse.

Step d) is preferably also performed by a central data processing device. This can moreover be identical with a data processing device in a hospital department on which tasks are to be executed.

### BRIEF DESCRIPTION OF THE DRAWINGS

Example embodiment variants of the invention are described below with reference to the drawing, in which:

FIG. 1 schematically depicts the organizational structure in a hospital, as required for the method according to an embodiment of the invention,

FIG. 2 is a flowchart representing how it can be made possible to coordinate the activity carried out by hospital departments by generating software and data,

FIG. 3 schematically depicts how a software system can be built which enables activity carried out in departments of a hospital to be coordinated, and

FIG. 4 is a flowchart representing the sequence of steps according to one embodiment variant of a method according to the invention.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

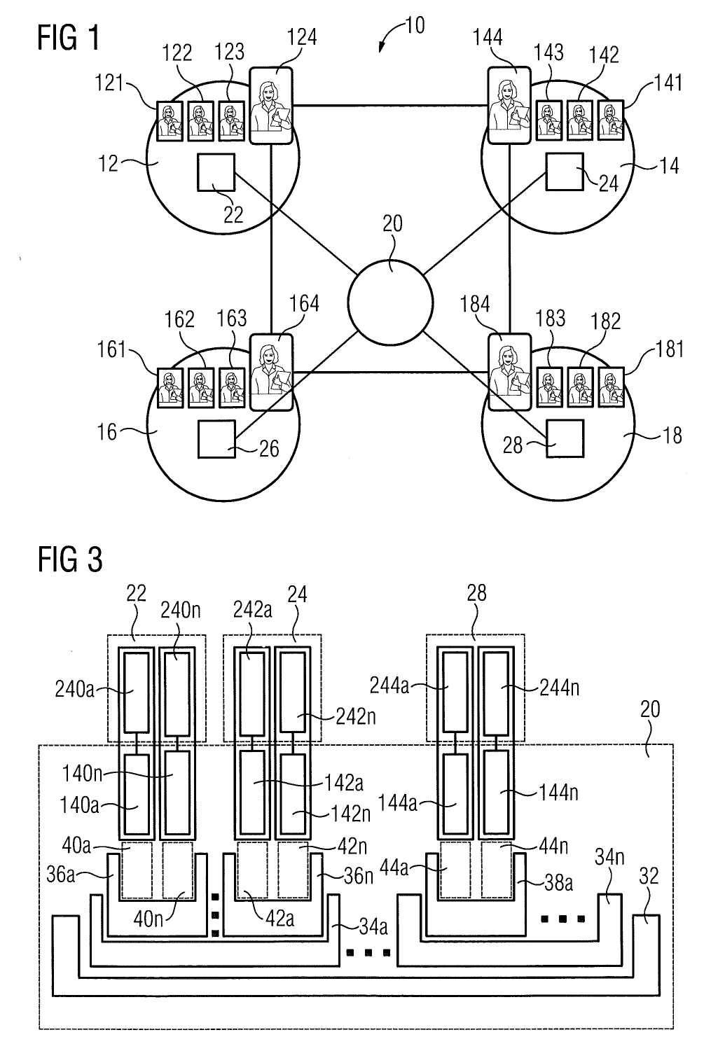

In a hospital 10 there are departments as shown schematically in FIG. 1, e.g. cardiology 12, radiology 14, pathology 16 and oncology 18. Cardiology 12 has treating physicians 121, 122, 123, 124, radiology has treating physicians 141, 142, 143, 144, pathology has treating physicians 161, 162, 163, 164, and oncology has treating physicians 181, 182, 183, 184.

It can now happen that a particular patient has to pass through several of these departments or through all of the departments. In the case of a brain disorder, for example, it is theoretically possible that cardiology will check whether the brain is being adequately supplied with blood by the heart, radiology will record images of the brain, pathology will investigate whether the lungs are absorbing enough oxygen, and finally in oncology the head will be examined using ultrasound, in particular to determine whether a tumor is present.

Within the framework of an embodiment of the present invention a so-called virtual team is formed in order to deal with a patient. Departmental teams consist of physicians all belonging to the same department. A virtual team is characterized in that it has at least two physicians from different departments. It is shown symbolically in FIG. 1 that the physicians 124, 144, 164 and 184 are to be combined in order to form a virtual team.

A central data processing device is required in order to manage a virtual team; this device is shown in FIG. 1 and denoted by 20. Connected to the central data processing device 20 are departmental data processing devices, in the present example one in each department. These departmental data processing devices are denoted by 22, 24, 26 and 28 in FIG. 1.

Embodiments of the present invention not only enable new situations to be defined in which virtual teams are to be formed, but also permit recourse to be made to schemes. In the present context reference is made to a “treatment situation”, which is to say that a patient is in a situation in which a specific treatment is necessary. By treatment is meant the totality of all activities that are to be provided in individual departments. Initially this mainly includes an examination of the patient for the purpose of creating the possibility of producing a diagnosis.

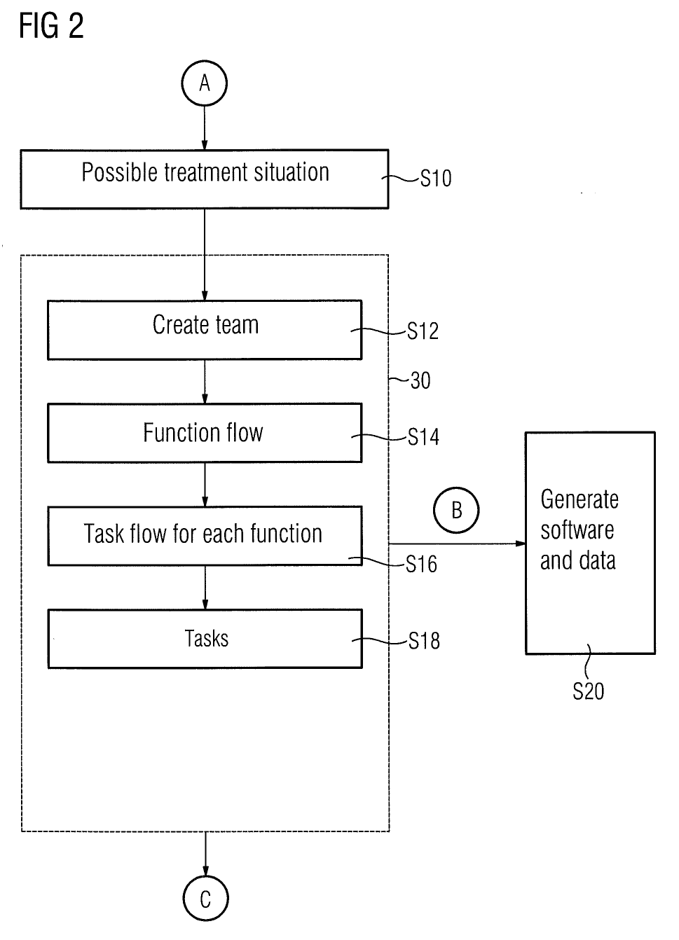

In the following it is explained with reference to FIG. 2 how preparations can be made for coordinating software-assisted activity of the individual hospital departments 12, 14, 16 and 18. In this regard reference is made to FIG. 2. The method starts at the point denoted by “A”: In step S10 a possible treatment situation is input, e.g. on the central data processing device 20. Software which provides a predefined input scheme 30 is deployed on the data processing device in which the input is made. Said scheme, which can also be designated in technical terminology as a “template”, includes that initially it is specified in relation to a possible treatment situation, what type of physician is to be part of the necessary virtual team. This takes place within the framework of step S12.

In the above-cited example of a malfunction of the brain (“mental disorder”) a virtual team appropriate to this possible treatment situation is created which comprises the roles of a cardiologist, a radiologist, a pathologist and an oncologist for different functions. Initially, rather than the role being filled by an actual individual, it is specified abstractly what purpose this individual has to fulfill. After step S12 a function flow is then specified (step S14), this being supported by the software scheme 30. In the case of the brain malfunction it is therefore specified that a cardiologist must record a two-dimensional image of the heart, must analyze same and write a report relating thereto. The next function entails a radiologist acquiring a 3D image dataset of the brain, selecting same and writing a report relating thereto. The next function entails a pathologist acquiring a two-dimensional image of the lungs, evaluating same and writing a report relating thereto. The last function comprises an oncologist examining the patient's head using ultrasound, forming an image of the patient and writing a report relating thereto.

In the course of performing the individual functions the treating physicians need to be supported by software. In this scenario the software takes action to ensure individual tasks are performed. Typically, a function entails that more than one individual task is required to be performed. For that reason a task flow is specified for each function in step S16. While the concept of the “function” abstractly includes the activity of the treating physician, what is understood by the concept of a “task” is that certain functionalities are handled by software.

In step S18 the individual tasks are then made available in the form of software. Either these are programmed directly or specified by provision of a so-called configuration file for tasks, or alternatively it is possible to resort to existing software.

In order to generate a software system, the method terminates via point “B” with step S20. In addition to the software, data is made available simultaneously, specifically in a file which contains the composition of the team appropriate to a possible treatment situation and in a file containing the function sequence and in one or more files containing task flow(s).

In this way software is obtained in a scheme as represented in FIG. 3.

The lowest level of the software is the operating system (referred to as the “site engine”) for the central data processing device 20, which operating system is denoted by 32 in FIG. 3. Running on top of the operating system is software 34a to 34n, each of which effects a coordination according to a function flow specified in step S14 specifically for a possible treatment situation. For each function from the function flow, software is then provided which monitors the task sequence (task flow). This software is denoted by 36a to 36n and 38a in FIG. 3. In turn the software 36a now enables tasks to execute, to which end corresponding software 40a, 40n, 42a, 42n, 44a, 44n is provided for each task.

The software modules 34a to 34n, 36a to 36n, 38a, 40a, 40n, 42a, 42n, 44a, 44n can each be designated as “containers”, a container being defined by the fact that it images program code into the working memory, in other words performs the operation termed “mapping”. For example, a task container 40a uses a task configuration file in order to generate a task, that is to say to make it executable in the working memory.

Corresponding software is available for each task, the software being subdivided in the present instance into so-called “frontend” software and “backend” software. In this arrangement the backend software is intended to run on the central data processing device 20, while the frontend software executes on the departmental data processing devices 22, 24 and 28. In the present example, backend software 140a associated with the task generated by the task container 40a is stored on the central data processing device 20, while associated frontend software 240a resides on the data processing device 22 of the cardiology department. The same applies to the software modules 140n, 240n, 142a, 242a, 142n, 242n, 144a, 244a or, as the case may be, 144n and 244n, the numbers between 140 and 150 in each case denoting backend software packages and the numbers between 240 and 250 denoting frontend software packages. The association between the individual software modules and the data processing devices 20, 22, 24 and 28 is illustrated by the dashed representation in FIG. 3.

The subdivision of the software associated with the tasks into backend software and frontend software enables one and the same task to be generated simultaneously by the central data processing device 20 and one of the departmental data processing devices 22, 24, 26 and 28, with the software-based user interface in particular being provided in the individual departments, while lower-priority activities are performed by the central data processing device.

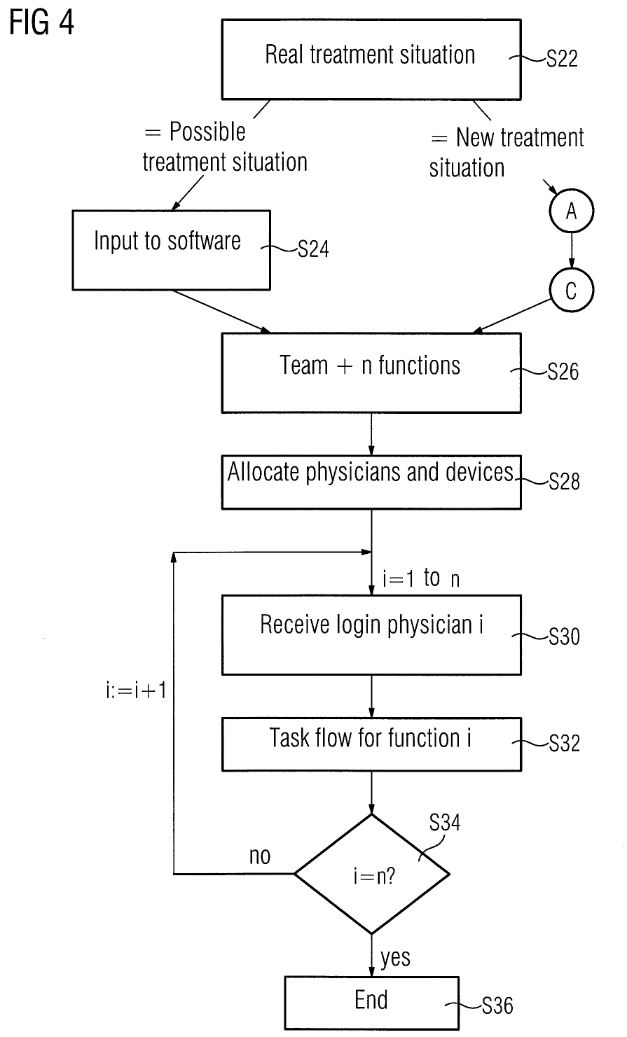

In the following it will now be explained with reference to FIG. 4, how a real patient can be treated.

The method of an embodiment according to FIG. 4 starts with the occurrence of a real treatment situation in step S22.

If the real treatment situation is one of the possible treatment situations for which software according to the scheme from FIG. 3 was previously created in step S20 of the method according to FIG. 2, then in the patient admissions unit an input appropriate to the existing software needs to be made only in step S24 in order for the software to identify one of the stored possible treatment situations as the real treatment situation.

In the event that the real treatment situation is none of the previously provided possible treatment situations, the method explained with reference to FIG. 2 is effectively reiterated, i.e. starting at point “A” and stopping at point “C”. It should be pointed out that a point “C” is named intentionally, and not point “B”, for if a new treatment situation arises, it is possible, when steps S12 to S18 are being performed, to fall back on what already exists in the software generated in step S20. Thus, for example, it may be that in a new treatment situation the same team has to be created as in a previously known possible treatment situation, and that said team also has to perform the same functions.

It may also be that a function flow specified for a possible treatment situation simply needs to be modified. If individual functions of the function flow for the new treatment situation are known, it is also not necessary to generate the associated task flow from new. Finally, the tasks also do not have to be generated from new each time. The method explained with reference to FIG. 2 is therefore exited at point “C” at any position, whether starting from step S12, starting from step S14, starting from step S16 or starting from step S18, or whether it be that individual steps (S14, S16) are effectively skipped or modified.

The end result is that, as in accordance with step S20, software with associated data is then available. No input as in step S24 is necessary, because it is self-evident in any event as a result of the new input that the possible treatment situation is identical to the real treatment situation which is the starting point for the method according to FIG. 4.

In the two above-discussed scenarios a team can now be assigned to the real treatment situation, as well as n functions of a function flow (step S26), it being possible to resort to the team created according to step S12. Step S26 therefore includes more a formal allocation of the team and the functions. In the following step S28, the individual roles of the team are now filled by attendant physicians, and devices are allocated at the same time. The allocation can be made in particular in such a way that the patient can be treated on a just-in-time basis, in other words has no waiting times. The allocated physicians can be selected so that they are available according to a timeline precisely when the function that is to be fulfilled by them is pending. The same applies to the availability of the devices.

The central data processing device 20 now puts itself into a state of readiness to receive a login. In this state it starts with the first function and waits for the login of the physician assigned to the first function. The physician can log in under a specific role. This can also happen implicitly in that the physician logs in at a specific departmental data processing device 22, 24, 26, 28, i.e. at the device in his/her department. If, according to step S30, such a login is now received, then according to step S32 the central data processing device ensures that the task flow associated with the respective function is executed. In the process the respective backend software (units 140a, 140n, . . . ) interacts with the respective frontend software (240a, 240n, . . . ).

In the example case of a brain malfunction, the login of the cardiologist 124 is received for the first function in step S30 and then a task flow specifically defined for the cardiologist is performed in step S32, enabling the cardiologist to record images of the heart and supporting him/her in the diagnosis and in the preparation of a medical report. For the second function, the login of the radiologist 144 is received in step S30 and a task flow appropriate thereto is performed in step S32 so that the radiologist 144 can record three-dimensional images of the patient's brain, can analyze said images and can write a medical report relating thereto. In the case of the third function, the login of the pathologist 164 is awaited in step S30 and by execution of a corresponding task flow according to step S32 it is made possible for said physician to record two-dimensional images of the lungs, to analyze same and to produce a report relating thereto. Finally, for the fourth function, the login of the oncologist 184 is awaited according to step S30 and in step S32 a task flow appropriate thereto is allowed to run, thereby assisting the oncologist 184 in acquiring ultrasound information about the patient's brain.

Steps S30 and S32 are performed for each of the n functions, this being illustrated in FIG. 4 by way of the formal step S34, according to which a check is made to determine whether a counter i has reached the number n, said counter being incremented by one and steps S30 and S32 being performed until finally the n functions have been processed and the method has been terminated according to step S36.

The invention provides an innovative concept whereby through formation of virtual teams in hospitals, initially through allocation of roles and then through allocation of specialist physicians to the roles, the treatment of a patient in a real treatment situation can be coordinated on an interdepartmental basis and available devices can be assigned in each case. It is possible in particular to carry out optimal time management so that the patient is treated as quickly as possible, without long waiting times. At the same time the use of expensive devices can be managed centrally and therefore organized in an optimal manner. The physicians' time can be optimally allocated. A physician may be a member of a plurality of virtual teams.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### LIST OF REFERENCE SIGNS

10 Hospital
12 Cardiology
14 Radiology
16 Pathology
18 Oncology
121, 122, 123, 124 Physicians of the cardiology department
141, 142, 143, 144 Physicians of the radiology department
161, 162, 163, 164 Physicians of the pathology department
181, 182, 183, 184 Physicians of the oncology department
20, 22, 24, 26, 28, 32 Data processing devices
30 Input scheme
34a to 34n, 36a to 36n, Software
38a, 40a, 40n, 42a,
42n, 44a, 44n
140a, 140n Backend software
240a, 240n Frontend software
i Counter
n Functions
S10, S12, S14, S16, Steps
S18, S20, S22, S24,
S26, S28, S30, S32,
S34, S36

What is claimed is:

1. A method for coordinating software-assisted activity of a plurality of departments of an organization, comprising:
a) providing a central data processing device and, for the plurality of departments, at least one departmental data processing device coupled to the central data processing device for data exchange purposes;
b) receiving at least one input for designating a possible starting situation;
c) receiving at least one input for specifying a function flow and roles assigned by a plurality of individual functions of the function flow;
d) receiving at least one input for assigning software task flows for each of the plurality of individual functions and providing the software for the software task;
e) if all inputs appropriate to a real starting situation that has currently occurred were made and the results of the inputs were made available to the central data processing device, equating a real starting situation to the possible starting situation and transitioning to step f), or
if steps a) to d) were carried out spaced apart in time prior to the occurrence of a real starting situation which is a possible starting situation, providing the results of the inputs on the central data processing device, receiving an input for designating the real starting situation, assigning the inputs made in relation to the corresponding possible starting situation to the real starting situation and transitioning to step f); and
f) for each function in the function flow assigned to the real starting situation: putting the central data processing device into a state of readiness to receive an identification input for the role assigned to the function at a departmental data processing device associated with said role and receiving such an identification input as well as generating the tasks associated with the function and executing the task flow using program code stored on memory of the central data processing device.

2. The method as claimed in claim 1, wherein, in step f), the tasks are generated through interaction between the central data processing device and a departmental data processing device associated with the respective role, the tasks at least one of acting on an output device connected to the departmental data processing device and responding to inputs made on an input device connected to the departmental data processing device.

3. The method as claimed in claim 1, wherein, in step e), the central data processing device assigns at least one of actual individuals and devices to the individual roles with a time interval for fulfilling the associated function in order to specify a timeline.

4. The method as claimed in claim 3, wherein, in step f), the central data processing device effects the issuing of notifications by the departmental data processing devices to actual individuals at least one of simultaneously and at times defined by the timeline.

5. A method for coordinating software-assisted activity of a plurality of hospital departments, comprising:
a) specifying a treatment situation from a plurality of stored possible treatment situations based on an input;
b) allocating a function flow with roles associated with each function;
c) allocating software task flows associated with each function; and
d) effecting the execution of tasks on data processing devices in hospital departments differing according to role in accordance with the function flow allocated to the treatment situation and software task flows assigned to the functions from said function flow.

6. The method as claimed in claim 2, wherein, in step e), the central data processing device assigns at least one of actual individuals and devices to the individual roles with a time interval for fulfilling the associated function in order to specify a timeline.

7. The method as claimed in claim 6, wherein, in step f), the central data processing device effects the issuing of notifications by the departmental data processing devices to actual individuals at least one of simultaneously and at times defined by the timeline.

8. A computer readable medium including program segments for, when executed on a computer device, causing the computer device to implement the method of claim 1.

9. A computer readable medium including program segments for, when executed on a computer device, causing the computer device to implement the method of claim 5.
